(19) United States
(12) Patent Application Publication
(10) Pub. No.: US 2026/0000834 A1
(43) Pub. Date: Jan. 1, 2026

(54) A sub-assembly of a medicament delivery device

(71) Applicant: SHL Medical AG, Zug (CH)

(72) Inventors: Oscar Alexandersson, Nacka Strand (SE); Anders Boström, Nacka Strand (SE)

(21) Appl. No.: 18/880,955

(22) PCT Filed: Jun. 26, 2023
(86) PCT No.: PCT/EP2023/067218

§ 371 Date: Jan. 3, 2025

(30) Foreign Application Priority Data
Jul. 7, 2022 (EP) 22183662.0

(51) Int. Cl. A61M 5/24; A61M 5/31; A61M 5/32

(52) U.S. Cl. CPC A61M 5/24; A61M 5/3135; A61M 5/3202

(57) ABSTRACT

The present disclosure provides a sub-assembly of a medicament delivery device, wherein the medicament delivery device comprises a medicament container, wherein the medicament container comprises a barrel extending along a longitudinal axis between a proximal end and a distal end, a medicament delivery member attached to the proximal end of the barrel, and a delivery member cap that encloses the medicament delivery member. The sub-assembly includes a delivery member guard comprising a body extending along the longitudinal axis between a proximal end and a distal end. The body is configured to at least partially surround the medicament container. The sub-assembly further includes a cap comprising a distally extending arm extending towards the distal end of the delivery member guard. The cap is removable from the delivery member guard by axially moving the cap relative to the delivery member guard from a distal position to a proximal position. The distally extending arm is configured to be deformed radially outward from the longitudinal axis by the barrel of the medicament container when the cap is in the distal position.

### CROSS-REFERENCE TO RELATED APPLICATIONS

The present application is a U.S. National Phase Application pursuant to 35 U.S.C. § 371 of International Application No. PCT/EP2023/067218 filed Jun. 26, 2023, which claims priority to EP patent application Ser. No. 22/183,662.0 filed Jul. 7, 2022. The entire disclosure contents of these applications are herewith incorporated by reference into the present application.

### TECHNICAL FIELD

The present disclosure generally relates to a sub-assembly of a medicament delivery device, and particularly to a sub-assembly of a medicament delivery device comprising a delivery member guard and a cap.

### BACKGROUND

Medicament delivery devices such as pen-type manual injectors or auto-injectors are generally known for the self-administration of a medicament by patients without formal medical training. For example, patients suffering from diabetes may require repeated injections of insulin, or patients may require regular injections of other types of medicaments such as a growth hormone.

Reducing the cost of raw materials of medicament delivery devices can reduce the cost of each medicament delivery device and thus make the medicament delivery device affordable for more patients.

Therefore, a medicament delivery device that is made of more plastic components than metal components is desirable as the metal components usually cost more than plastic components. Furthermore, a medicament delivery device that is easy to be assembled, e.g., requires fewer assembling tools involved, can also reduce the cost of assembling.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device. The medicament delivery device comprises a medicament container. The medicament container comprises a barrel extending along a longitudinal axis between a proximal end and a distal end, a medicament delivery member attached to the proximal end of the barrel, and a delivery member cap that encloses the medicament delivery member. The sub-assembly comprises: a delivery member guard comprising a body extending along the longitudinal axis between a proximal end and a distal end; wherein the body is configured to at least partially surround the medicament container; and a cap comprising a distally extending arm extending towards the distal end of the delivery member guard. The distally extending arm comprises a protrusion extending towards the longitudinal axis. The body of the delivery member guard comprises a support surface facing toward the longitudinal axis. The distally extending arm is adjacent to the support surface in a direction transverse to the longitudinal axis. The distally extending arm is configured to be adjacent to the medicament container in a direction transverse to the longitudinal axis when the medicament container is assembled to the sub-assembly. The cap is removable from the delivery member guard by axially moving the cap relative to the delivery member guard in the distal direction so that the protrusion is engaged with the delivery member cap when the medicament container is positioned in the sub-assembly.

In a medicament delivery device comprises the sub-assembly as mentioned above, the protrusion is configured to grip the delivery member cap when the protrusion moved distally to be aligned with the support surface in the direction transverse to the longitudinal axis such that the delivery member cap can be removed. The protrusion may not initially engage with the delivery member cap as the distally extending arm is pressed radially outward by the barrel of the medicament container. Therefore, the sub-assembly can be suitable for different size of medicament containers and/or different length of delivery member caps. Furthermore, as the cap is not engaged with the delivery member cap before cap removal, delivery member cap will not accidentally be pulled off from the medicament container when the medicament delivery device is dropped before use.

Preferably, according to another embodiment, the protrusion of the distally extending arm is configured to engage with the barrel of the medicament container when the cap is in the distal position and is configured to engage with the delivery member cap when the cap is in the proximal position.

Preferably, according to another embodiment, the protrusion of the distally extending arm comprises a proximally directed surface. The proximally directed surface is generally perpendicular to the longitudinal axis.

Preferably, according to another embodiment, the protrusion of the distally extending arm comprises a distally directed surface. The distally directed surface is bevelled to the longitudinal axis.

Preferably, according to another embodiment, the sub-assembly comprises a housing extending along the longitudinal axis between a proximal end and a distal end. The housing is configured to accommodate the medicament container. The proximal end of the delivery member guard telescopically protrudes from the proximal end of the housing.

Preferably, according to another embodiment, the cap is removably attached to the proximal end of the housing.

Preferably, according to another embodiment, the cap is attached to the housing via a snap-fit engagement.

Preferably, according to another embodiment, the cap comprises a tubular body. The tubular body of the cap at least partially surrounds the proximal end of the housing.

Preferably, according to another embodiment, the housing comprises a protrusion extending from the proximal end of the housing in the direction transverse to the longitudinal axis. A cut-out is arranged in a wall of the tubular body of the cap. The protrusion of the housing is at least partially positioned within the cut-out of the tubular body of the cap when the cap is in the distal position.

Preferably, according to another embodiment, the tubular body of the cap comprises a tubular section and a transverse section. The tubular section extends along the longitudinal axis between a proximal end and a distal end. The transverse section extends from the proximal end of the tubular section in the direction transverse to the longitudinal axis. The distally extending arm extends from the transverse section towards the distal end of the delivery member guard.

Preferably, according to another embodiment, the distally extending arm comprises a second protrusion extending towards the delivery member guard. The second protrusion defined a proximally directed surface. The proximally directed surface is bevelled to the longitudinal axis.

Preferably, according to another embodiment, the body of the delivery member guard comprises a tubular wall and a side wall. The tubular wall is configured to surround the medicament delivery member. The tubular wall extends along the longitudinal axis between the proximal end of the body of the delivery member guard and the side wall.

Preferably, according to another embodiment, a recess/cut-out is in the side wall. The recess/cut-out is next to the support surface which is defined by the side wall in the direction of the longitudinal axis. The second protrusion of the distally extending arm is positioned within the recess/cut-out when the cap is in the distal position.

Preferably, according to another embodiment, the distally extending arm is made of plastic.

Another aspect of the invention provides a sub-assembly of a medicament delivery device. The medicament delivery device comprises a medicament container. The medicament container comprises a barrel extending along a longitudinal axis between a proximal end and a distal end, a medicament delivery member attached to the proximal end of the barrel, and a delivery member cap that encloses the medicament delivery member. The sub-assembly comprises: a delivery member guard comprising a body extending along the longitudinal axis between a proximal end and a distal end; wherein the body is configured to at least partially surround the medicament container; and a cap comprising a distally extending arm extending towards the distal end of the delivery member guard. The cap is removably connected to the delivery member guard. The distally extending arm is configured to flex radially outward when the cap is connected to the delivery member guard. The distally extending arm is configured to be pressed radially inwardly to engage with the delivery member cap by the body of the delivery member guard during the cap removal.

Preferably, according to another embodiment, the distally extending arm is configured to be pushed radially inwardly to positioned within a gap between the barrel and the delivery member cap.

Another aspect of the invention provides a medicament delivery device comprising the sub-assembly as mentioned above. The medicament delivery device comprises a medicament container. The medicament container comprises a barrel extending in the longitudinal axis between a proximal end and a distal end, a medicament delivery member attached to the proximal end of the barrel, and a delivery member cap enclosed by the medicament delivery member.

Preferably, according to another embodiment, the medicament delivery member is an injection needle, an infusion catheter, or a spray nozzle.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier.

Pharmaceutical formulations comprising a drug as listed herein (or a pharmaceutically acceptable salt of the drug) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIGS. 1A-1B schematically show cross-section views of a sub-assembly of the invention when a cap is in a distal position and the cap is in a proximal position respectively.

FIG. 2 schematically shows a perspective view of the cap of the sub-assembly of FIG. 1.

FIG. 3 schematically shows a perspective view of a delivery member guard of the sub-assembly of FIG. 1.

FIG. 4 schematically shows a perspective view of the cap of FIG. 2 and the delivery member guard of FIG. 3.

FIGS. 5-6 schematically show perspective views of a housing of the sub-assembly of FIG. 1 with the cap of FIG. 2 and the delivery member guard of FIG. 3.

FIGS. 7-9 schematically show perspective views of the sub-assembly of FIG. 1 in another example.

### DETAILED DESCRIPTION

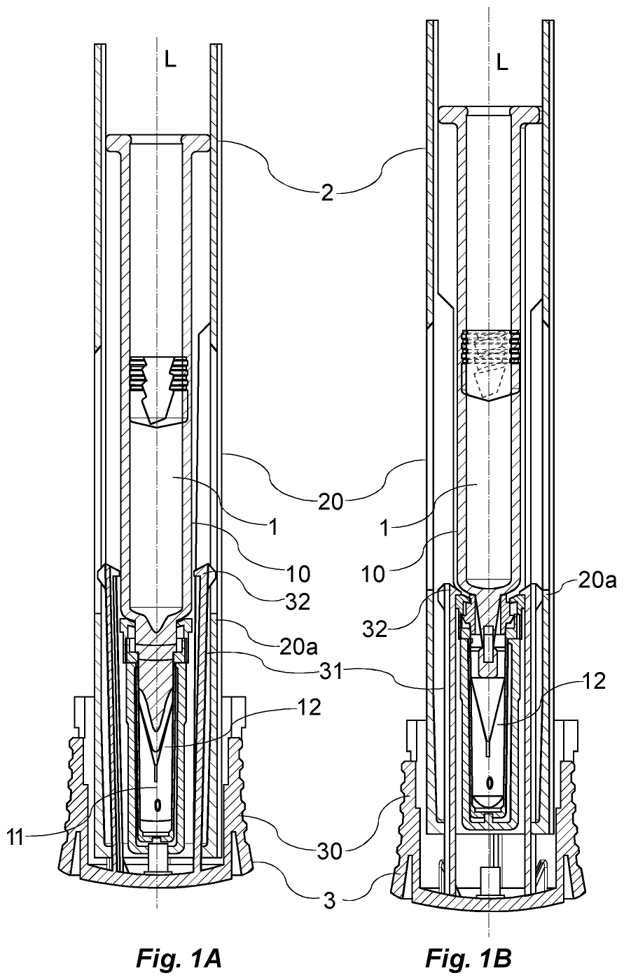

FIGS. 1-9 illustrate a sub-assembly of a medicament delivery device. The medicament delivery device comprises a medicament container 1. The medicament container 1 comprises a barrel 10 extending along a longitudinal axis L between a proximal end and a distal end, a medicament delivery member 11 attached to the proximal end of the barrel 10, and a delivery member cap 12 that encloses the medicament delivery member 11, as shown in FIGS. 1A-1B. In one example, the medicament container is a syringe. Additionally, in another example, the medicament delivery member is a needle. Additionally, in another example, the delivery member cap is a rigid needle shield (RNS) and/or a flexible needle shield (FNS). In a preferred example, the medicament container comprises a flange extending around the distal end of the medicament container. Additionally, in a preferred example, the medicament delivery device comprises a container collar configured to engage with a proximally directed surface of the flange of the medicament container. In a preferred example, the medicament container comprises a housing accommodating the medicament container and the container collar. In a preferred example, the medicament container is fixed to the housing by the container collar such that any proximal movement of the medicament container is prevented by the container collar. In a preferred example, the container collar is attached to the housing by a snap-fit attachment. The container collar can be formed in ring shape or c-clip. The container collar can be made of elastic material.

In another preferred example, the medicament delivery device comprises a distal container block configured to be in contact with a distally directed surface of the flange of the medicament container. The container block can be one or more flexible arm or a plastic spring structure. In this example, the deformation of the container block can be used to compensate the length variation of the medicament container (due to the engineering tolerance, e.g., containers made from mass production might have 5-10% length variation). In a preferred example, the container block is fixed to the housing, e.g., as an integral part of the housing or immovably attached to the housing.

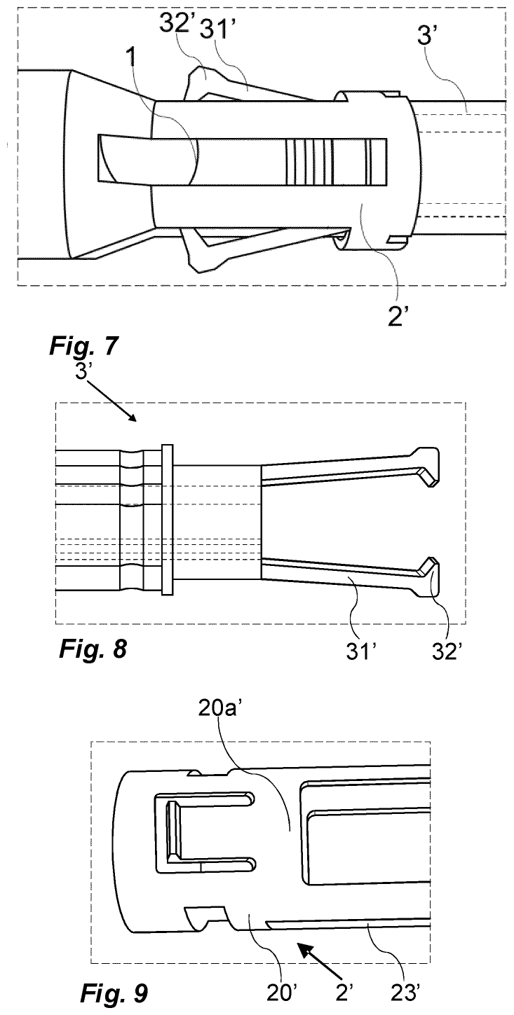

The sub-assembly comprises a delivery member guard 2; 2′ and a cap 3; 3′. The delivery member guard 2; 2′ comprising a body 20; 20′ extending along the longitudinal axis L between a proximal end and a distal end. The body 20; 20′ is configured to at least partially surround the medicament container 1, as shown in FIGS. 1A-1B, and FIG. 7. In one example, the body 20; 20′ of the delivery member guard 2; 2′ comprises a tubular wall 22 and a side wall 21, as shown in FIG. 4. The tubular wall 22 is configured to surround the medicament delivery member. In this example, the tubular wall 22 extends along the longitudinal axis L between the proximal end of the body 20; 20′ of the delivery member guard 2; 2′ and the side wall 21. In this example, the tubular wall 21 is configured to cover the medicament delivery member to prevent the medicament delivery member from being contacted by a user so that a potential injury or contamination of the delivery member can be prevented. In one example, the side wall is defined by a distally extending arm.

The cap 3; 3′ is removably connected to the delivery member guard 2; 2′. The cap 3; 3′ comprises a distally extending arm 31; 31′ extending towards the distal end of the delivery member guard 2; 2′. The distally extending arm 31; 31′ comprises a protrusion 32; 32′ extending towards the longitudinal axis L. The body 20; 20′ of the delivery member guard 2; 2′ comprises a support surface 20a; 20a′ facing towards the longitudinal axis L. The distally extending arm 31; 31′ is configured to be positioned between the support surface 20a; 20a′ and the medicament container 1 in a direction transverse to the longitudinal axis L when the medicament container 1 is assembled to the sub-assembly and the cap 3; 3′ is connected to the delivery member guard 2; 2′. The support surface 20a; 20a′ is configured to press the distally extending arm 31; 31′ radially inward toward the longitudinal axis L so that the protrusion 32; 32′ can tightly grip on the delivery member cap during the removal of the cap 3; 3′. As a result, the removal of the cap can also remove the delivery member cap from the medicament container 1. In a preferred example, the delivery member guard 2; 2′ comprises a support wall having the support surface.

The distally extending arm 31; 31′ is configured to flex radially outward when the cap 3; 3′ is connected to the delivery member guard 2; 2′. The distally extending arm 31; 31′ is configured to be pressed radially inwardly to engage with the delivery member cap by the body of the delivery member guard 2; 2′ during the cap 3; 3′ removal.

In a preferred example, the cap is snap-fitted to the housing of the medicament delivery device. In this example, as the distally extending arm is not engaged with the delivery member cap, when the user plans to remove the cap from the housing, the user only needs to overcome the resistance that is generated from the snap-fit between the cap and the housing. Once the cap is detached from the housing, the distally extending arm moved in the proximal direction together with the cap, the distally extending arm is pressed radially inwardly to engage with the delivery member cap, then the further proximal movement of the cap results in that the delivery member cap being detached. As the friction between the delivery member cap and the delivery member can be high, the user might have a difficulty to overcome the friction and the snap-fit between the cap and the housing; however, with the above-mentioned design, it is easier for the user to remove the cap completely from the housing of the medicament delivery device.

In a preferred example, the cap 3; 3′ is removable from the delivery member guard 2; 2′ by axially moving the cap 3; 3′ relative to the delivery member guard 2; 2′ from a distal position where the support surface 20a; 20a′ is spaced apart from the protrusion 32; 32′ of the distally extending arm 31; 31′ in the direction transverse to the longitudinal axis L, as shown in FIG. 1A and FIG. 7, to a proximal position where the support surface 20a; 20a′ is aligned with the protrusion 32; 32′ of the distally extending arm 31; 31′ in the direction transverse to the longitudinal axis L, as shown in FIG. 1B. The distally extending arm 31; 31′ is configured to be deformed radially outward from the longitudinal axis L by the barrel 10 of the medicament container 1 when the cap 3; 3′ is in the distal position.

In a preferred example, the protrusion 32; 32′ of the distally extending arm 31; 31′ is configured to engage with the barrel 10 of the medicament container when the cap is in the distal position, as shown in FIG. 1A and FIG. 7, and is configured to engage with the delivery member cap when the cap is in the proximal position, as shown in FIG. 1B. In a preferred example, the protrusion 32; 32′ is configured to engage with a distal end of the delivery member cap. Alternatively, the protrusion is configured to engage with a side surface of the delivery member cap. In one example where there is a gap between the distal end of the delivery member cap and the barrel 10 of the medicament container 1, when the cap is moved in the proximal direction relative to the medicament contain for being removed from the medicament container, the proximal movement of the cap causes the protrusion to move into the gap. As a result, the protrusion abuts the distal end of the delivery member cap. Further, the cap removal movement causes the protrusion to remove the delivery member cap from the medicament container 10. Alternatively, in one example where the distal end of the delivery member cap and the barrel 10 of the medicament container 1 are in contact or partially overlap, the cap removal movement causes the protrusion to push on the delivery member cap until a gap between the distal end of the delivery member cap and the barrel 10 of the medicament container 1 is made, thus, the protrusion abuts the distal end of the delivery member cap. Further, the cap removal movement causes the protrusion to remove the delivery member cap from the medicament container 10.

Alternatively, in another example, the distally extending arm is configured to be flexed radially outwardly directly by the barrel of the medicament container. In other word, the barrel of the medicament container will contact a side surface of the distally extending arm, and thereby flexes the distally extending arm with the protrusion being radially spaced apart from the barrel of the medicament container.

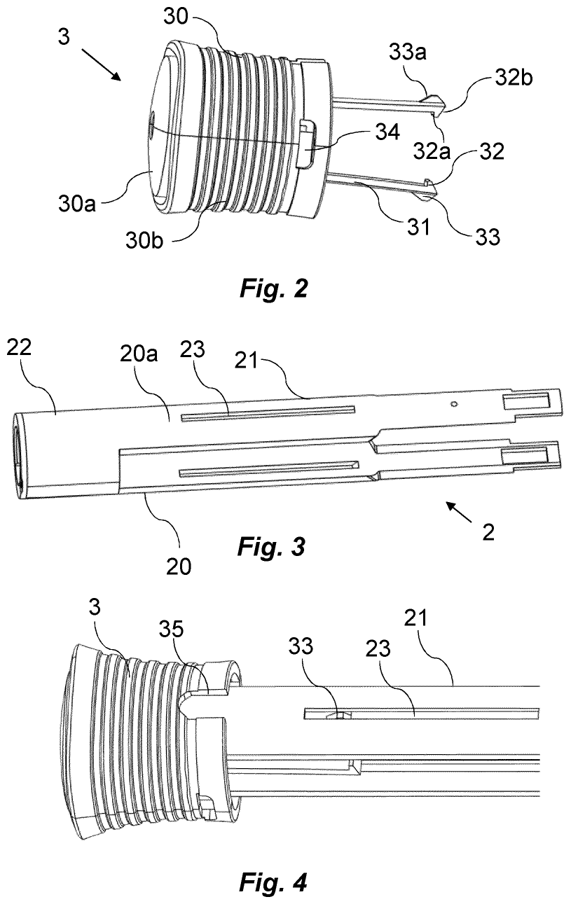

In a preferred example, the protrusion 32 of the distally extending arm 31 comprises a proximally directed surface 32a, as shown in FIG. 2. The proximally directed surface 32a is generally perpendicular to the longitudinal axis L. In this example, the sub-assembly is more suitable for using the protrusion to grip on the distal end of the delivery member cap. Alternatively, the proximally directed surface is bevelled relative to the longitudinal axis L. In this example, the sub-assembly is more suitable for using the protrusion to grip on the side surface of the delivery member cap.

The cap 3 comprises a body 30. In a preferred example, the cap 3 comprises a tubular body 30, as shown in FIG. 2. In a preferred example, the tubular body 30 of the cap 3 comprises a tubular section 30b and a transverse section 30a. The tubular section 30 extends along the longitudinal axis L between a proximal end of the tubular section 30b and a distal end of the tubular section 30b. The transverse section 30a extends from the proximal end of the tubular section 30b in the direction transverse to the longitudinal axis L. In one example, the distally extending arm 31 extends from the transverse section 30a towards the distal end of the delivery member guard 2. Alternatively, the distally extending arm extends from an inner surface of the tubular section towards the distal end of the delivery member guard 2. Alternatively, the distally extending arm extends from the distal end of the tubular section. In a preferred example, the tubular body 30 of the cap 3 is configured to receive the delivery member cap.

In another example, the protrusion 32 of the distally extending arm 31 comprises a distally directed surface 32b. The distally directed surface 32b is bevelled relative to the longitudinal axis L. In this example, when the medicament container 1 is inserted into the cap, the delivery member cap is in contact with the distally directed surface 32b, and thus, deforms the distally extending arm 31 radially outward from the longitudinal axis L. As a result, the delivery member cap can enter the tubular body 30 of the cap 3.

In one example, the distally extending arm 31 comprises a second protrusion 33 extending towards the delivery member guard. The second protrusion 33 defined a proximally directed surface 33a. The proximally directed surface 33a is bevelled relative to the longitudinal axis L, as shown in FIG. 2. The second protrusion 33 is configured to engage with the support surface 20a of the delivery member guard 2, therefore, the protrusion 32 of the distally extending arm 31 can be pressed tightly to engage with the delivery member cap.

Additionally, in one example where the distally extending arm 31 comprises the second protrusion 33, a recess/cut-out 23 is in the side wall 21. The recess/cut-out 23 is next to the support surface 20a which is defined by the side wall 21 in the direction of the longitudinal axis L. In this example, the second protrusion 33 of the distally extending arm 31 is positioned within the recess/cut-out 23 when the cap 3 is in the distal position, as shown in FIGS. 3-4. In one example, the delivery member guard 2 comprises a window configured to be aligned with the medicament container so that the user can observe the medicament container via the window. In one example, the window is an opening in the side wall, or the window is a transparent part of the side wall. In one example, the window is the circumferentially offset to the recess/cut-out 23′, as shown in FIG. 7.

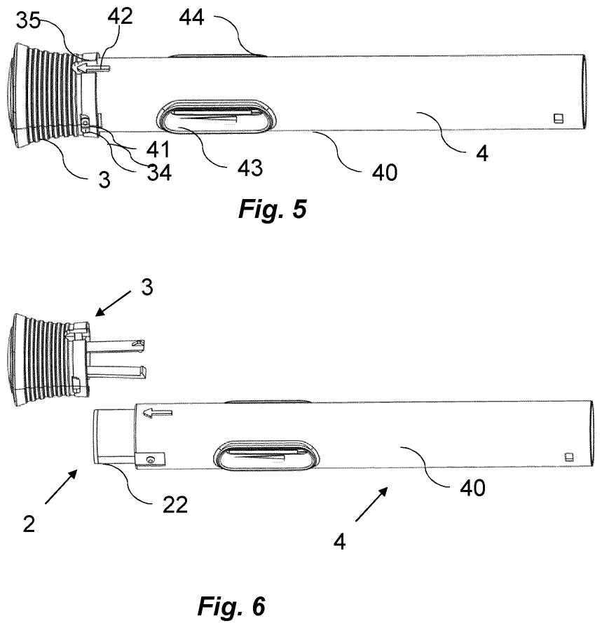

In another example, the sub-assembly comprises a housing 4 extending along the longitudinal axis L between a proximal end and a distal end. The housing 4 is configured to accommodate the medicament container 1. The housing 4 comprises a housing body 40. In one example, as shown in FIGS. 5-6, the housing body is tubular. The housing body 40 can be cylindrical, as most medicament containers are cylindrical, so that the medicament delivery device with the sub-assembly can be compact. Alternatively, the housing body comprises a non-circular cross-section when being observed along the longitudinal axis L, such as a triangle, rectangular or oval cross-section when being observed along the longitudinal axis L, so that the housing body will not roll on a flat surface. The housing body can be any suitable shape depending on the design, e.g., targeted patient groups.

In this example, the proximal end of the delivery member guard 2 telescopically protrudes from the proximal end of the housing 4, as shown in FIG. 6. In a preferred example, the cap 3 is removably attached to the proximal end of the housing 1, as shown in FIGS. 5-6. In a preferred example, the cap 3 is attached to the housing 1 via a snap-fit engagement.

Additionally, in one example where the cap 3 comprises the tubular body 30, the tubular body 30 of the cap 3 at least partially surrounds the proximal end of the housing 4. In one example, the housing 4 comprises a protrusion 41 extending from the proximal end of the housing 4 in the direction transverse to the longitudinal axis L. In this example, a cut-out 34 is arranged in a wall of the body 30 of the cap 3. In this example, the protrusion 41 of the housing 4 is at least partially positioned within the cut-out 34 of the body 30 of the cap 3 when the cap 3 is in the distal position, as shown in FIG. 5.

In another example, the housing 4 comprises a second protrusion 42 and a second cut-out 35 is arranged in a wall of the body 30 of the cap 3. In this example, the second protrusion 42 and the second cut-out 35 are configured to provide both visual and tactile indications to the user. For example, as shown in FIGS. 5-6, the user can see the second protrusion, e.g., as a proximally pointing arrow, that indicates the direction that the user can remove the cap. Furthermore, if the user accidentally twists the cap 3, the second protrusion will be blocked within the second cut-out. As a result, the user will know that the cap should not be twisted relative to the housing for the cap removal.

In another preferred example, the distally extending arm is made of plastic. In one example, the distally extending arm is integral to the cap.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are defined by the following clauses.
1. A sub-assembly of a medicament delivery device, wherein the medicament delivery device comprises a medicament container (l), and wherein the medicament container (l) comprises a barrel (10) extending along a longitudinal axis (L) between a proximal end and a distal end, a medicament delivery member (11) attached to the proximal end of the barrel (10), and a delivery member cap (12) that encloses the medicament delivery member (11), the sub-assembly comprising:
a delivery member guard (2) comprising a body (20) extending along the longitudinal axis (L) between a proximal end and a distal end; wherein the body (20) is configured to at least partially surround the medicament container (l);
a cap (3) comprising a distally extending arm (31) extending towards the distal end of the delivery member guard (2);
wherein the distally extending arm (31) comprises a protrusion (32) extending towards the longitudinal axis (L);
wherein the body (20) of the delivery member guard (2) comprises a support surface (20a) facing towards the longitudinal axis (L);
wherein the distally extending arm (31) is configured to be positioned between the support surface (20a) and the medicament container (l) in a direction transverse to the longitudinal axis (L) when the medicament container (l) is assembled to the sub-assembly;
wherein the cap (3) is removable from the delivery member guard (2) by axially moving the cap (3) relative to the delivery member guard (2) from a distal position where the support surface (20a) is not aligned with the protrusion (32) of the distally extending arm (31) in the direction transverse to the longitudinal axis (L) to a proximal position where the support surface (20a) is aligned with the protrusion (32) of the distally extending arm (31) in the direction transverse to the longitudinal axis (L) so that the protrusion is engaged with the delivery member cap when the medicament container is positioned in the sub-assembly; and
wherein the distally extending arm (31) is configured to be deformed radially outward from the longitudinal axis by the barrel (10) of the medicament container (l) when the cap (3) is in the distal position.
2. The sub-assembly according to clause 1, wherein the protrusion of the distally extending arm is configured to engage with the barrel of the medicament container when the cap is in the distal position and is configured to engage with the delivery member cap when the cap is in the proximal position.
3. The sub-assembly according to any one of the preceding clauses, wherein the protrusion of the distally extending arm comprises a proximally directed surface; and wherein the proximally directed surface is generally perpendicular to the longitudinal axis.
4. The sub-assembly according any one of the preceding clauses, wherein the protrusion of the distally extending arm comprises a distally directed surface; and wherein the distally directed surface is bevelled to the longitudinal axis.
5. The sub-assembly according to any of preceding clauses, the sub-assembly comprising a housing extending along the longitudinal axis between a proximal end and a distal end; wherein the housing is configured to accommodate the medicament container; and wherein the proximal end of the delivery member guard telescopically protrudes from the proximal end of the housing.
6. The sub-assembly according to clause 5, wherein the cap is removably attached to the proximal end of the housing.
7. The sub-assembly according to clause 6, wherein the cap is attached to the housing via a snap-fit engagement.
8. The sub-assembly according to any of clauses 5-7, wherein the cap comprises a tubular body; wherein the tubular body of the cap at least partially surrounds the proximal end of the housing.
9. The sub-assembly according to clauses 8, wherein the housing comprises a protrusion extending from the proximal end of the housing in the direction transverse to the longitudinal axis; wherein a cut-out is arranged in a wall of the tubular body of the cap; and wherein the protrusion of the housing is at least partially positioned within the cut-out of the tubular body of the cap when the cap is in the distal position.
10. The sub-assembly according to clauses 8 or 9, wherein the tubular body of the cap comprises a tubular section and a transverse section; wherein the tubular section extends along the longitudinal axis between a proximal end and a distal end; wherein the transverse section extends from the proximal end of the tubular section in the direction transverse to the longitudinal axis; and wherein the distally extending arm extends from the transverse section towards the distal end of the delivery member guard.
11. The sub-assembly according to any of the preceding clauses, wherein the distally extending arm comprises a second protrusion extending towards the delivery member guard; wherein the second protrusion defined a proximally directed surface; and wherein the proximally directed surface is bevelled to the longitudinal axis.
12. The sub-assembly according to any of the preceding clauses, wherein the body of the delivery member guard comprises a tubular wall and a side wall; wherein the tubular wall is configured to surround the medicament delivery member; wherein the tubular wall extends along the longitudinal axis between the proximal end of the body of the delivery member guard and the side wall.
13. The sub-assembly according to a combination of clause 11 and 12, wherein a recess/cut-out is in the side wall; wherein the recess/cut-out is next to the support surface which is defined by the side wall in the direction of the longitudinal axis; and wherein the second protrusion of the distally extending arm is positioned within the recess/cut-out when the cap is in the distal position.
14. The sub-assembly according to any of the preceding clauses, wherein the distally extending arm is made of plastic.
15. A medicament delivery device comprising the sub-assembly according to any of the preceding clauses; wherein the medicament delivery device comprises a medicament container; and wherein the medicament container comprises a barrel extending in the longitudinal axis between a proximal end and a distal end, a medicament delivery member attached to the proximal end of the barrel, and a delivery member cap enclosed the medicament delivery member.

1-15. (canceled)

16. A sub-assembly of a medicament delivery device, wherein the medicament delivery device comprises a medicament container, and wherein the medicament container comprises a barrel extending along a longitudinal axis between a proximal end and a distal end, a medicament delivery member attached to the proximal end of the barrel, and a delivery member cap that encloses the medicament delivery member, the sub-assembly comprising:
a delivery member guard comprising a body extending along the longitudinal axis between a proximal end and a distal end, wherein the body is configured to at least partially surround the medicament container; and
a cap comprising a distally extending arm extending towards the distal end of the delivery member guard,
wherein the distally extending arm comprises a protrusion extending towards the longitudinal axis,
wherein the body of the delivery member guard comprises a support surface facing towards the longitudinal axis, wherein the protrusion is distally arranged relative to the support surface,
wherein the distally extending arm is adjacent to the support surface in a direction transverse to the longitudinal axis, and wherein the distally extending arm is configured to be adjacent to the medicament container in a direction transverse to the longitudinal axis when the medicament container is assembled to the sub-assembly, and
wherein the cap is removable from the delivery member guard by axially moving the cap relative to the delivery member guard in the distal direction so that the protrusion is engaged with the delivery member cap when the medicament container is positioned in the sub-assembly.

17. The sub-assembly according to claim 16, wherein the distally extending arm is configured to be positioned between the support surface and the medicament container in a direction transverse to the longitudinal axis when the medicament container is assembled to the sub-assembly,
wherein the cap is removable from the delivery member guard by axially moving the cap relative to the delivery member guard from a distal position where the support surface is spaced apart from the protrusion of the distally extending arm in the direction transverse to the longitudinal axis to a proximal position where the support surface is aligned with the protrusion of the distally extending arm in the direction transverse to the longitudinal axis so that the protrusion is engaged with the delivery member cap when the medicament container is positioned in the sub-assembly, and
wherein the distally extending arm is configured to be deformed radially outward from the longitudinal axis by the barrel of the medicament container when the cap is in the distal position.

18. The sub-assembly according to claim 16, wherein the protrusion of the distally extending arm is configured to engage with the barrel of the medicament container when the cap is in the distal position and is configured to engage with the delivery member cap when the cap is in the proximal position.

19. The sub-assembly according to claim 16, wherein the protrusion of the distally extending arm comprises a proximally directed surface, and wherein the proximally directed surface is generally perpendicular to the longitudinal axis.

20. The sub-assembly according to claim 16, wherein the protrusion of the distally extending arm comprises a distally directed surface, and wherein the distally directed surface is bevelled to the longitudinal axis.

21. The sub-assembly according to claim 16, the sub-assembly comprising a housing extending along the longitudinal axis between a proximal end and a distal end, wherein the housing is configured to accommodate the medicament container, and wherein the proximal end of the delivery member guard telescopically protrudes from the proximal end of the housing.

22. The sub-assembly according to claim 21, wherein the cap is removably attached to the proximal end of the housing.

23. The sub-assembly according to claim 22, wherein the cap is attached to the housing via a snap-fit engagement.

24. The sub-assembly according to claim 21, wherein the cap comprises a tubular body, and wherein the tubular body of the cap at least partially surrounds the proximal end of the housing.

25. The sub-assembly according to claim 24, wherein the housing comprises a protrusion extending from the proximal end of the housing in the direction transverse to the longitudinal axis, wherein a cut-out is arranged in a wall of the tubular body of the cap, and wherein the protrusion of the housing is at least partially positioned within the cut-out of the tubular body of the cap when the cap is in the distal position.

26. The sub-assembly according to claim 24, wherein the tubular body of the cap comprises a tubular section and a transverse section, wherein the tubular section extends along the longitudinal axis between a proximal end and a distal end, wherein the transverse section extends from the proximal end of the tubular section in the direction transverse to the longitudinal axis, and wherein the distally extending arm extends from the transverse section towards the distal end of the delivery member guard.

27. The sub-assembly according to claim 16, wherein the distally extending arm comprises a second protrusion extending towards the delivery member guard, wherein the second protrusion defined a proximally directed surface, and wherein the proximally directed surface is bevelled to the longitudinal axis.

28. The sub-assembly according to claim 16, wherein the body of the delivery member guard comprises a tubular wall and a side wall, wherein the tubular wall is configured to surround the medicament delivery member, and wherein the tubular wall extends along the longitudinal axis between the proximal end of the body of the delivery member guard and the side wall.

29. The sub-assembly according to claim 28, wherein a recess/cut-out is in the side wall, wherein the recess/cut-out is next to the support surface which is defined by the side wall in the direction of the longitudinal axis, and wherein the second protrusion of the distally extending arm is positioned within the recess/cut-out when the cap is in the distal position.

30. The sub-assembly according to claim 16, wherein the distally extending arm is made of plastic.
